(19) United States
(12) Patent Application Publication
(10) Pub. No.: US 2012/0107423 A1
(43) Pub. Date: May 3, 2012

(54) Methods of Using Inhaled Nitric Oxide Gas for Treatment of Acute Respiratory Distress Syndrome

(75) Inventor: Brahm Goldstein, Annandale, NJ (US)

(73) Assignee: INO Therapeutics LLC, Hampton, NJ (US)

(21) Appl. No.: 13/283,759

(22) Filed: Oct. 28, 2011

Related U.S. Application Data
(60) Provisional application No. 61/408,810, filed on Nov. 1, 2010.

(51) Int. Cl. A61K 33/00; A61P 11/00

(52) U.S. Cl. 424/718

(57) ABSTRACT

The present invention provides a treatment of acute respiratory distress syndrome (ARDS) using short term dosing of nitric oxide.

### CROSS REFERENCE TO RELATED APPLICATIONS

This application claims priority under 35 U.S.C. §119(e) to U.S. Patent Application Ser. No. 61/408,810, filed Nov. 1, 2010, which is hereby incorporated by reference in its entirety.

### FIELD OF THE INVENTION

The present invention relates to methods of using inhaled nitric oxide gas to improve long-term pulmonary function in a subject with acute respiratory distress syndrome.

### BACKGROUND OF THE INVENTION

Assessment of treatments for acute respiratory distress syndrome (ARDS) has focused on short-term outcomes, most often mortality; little information exists regarding the long-term effects of ARDS treatment. Patients who survive an episode of ARDS may have long-term obstructive and restrictive pulmonary abnormalities, as well as, pulmonary gas exchange impairment. There is a need to evaluate inhaled nitric oxide effects on long-term pulmonary function in ARDS patients. The present invention is directed to the unexpected finding that short term treatment of ARDS using inhaled nitric oxide gas improves chronic pulmonary function in ARDS survivors.

### SUMMARY OF THE INVENTION

The present invention is directed to a method for treating a subject with impaired pulmonary function as a result of acute respiratory distress syndrome (ARDS) via administration of a low dose of inhaled nitric oxide (NO) wherein the inhaled NO improves pulmonary function after short term treatment.

The invention further provides for the administration of NO via inhalation. In one embodiment, the subject is treated at a dosage of about 5 ppm for up to 28 days.

### BRIEF DESCRIPTION OF THE DRAWINGS

FIG. 1 is a chart comparing the disposition of placebo versus control subjects (alive and off assisted breathing by day 28). The abbreviations in the chart include: AOAB=alive and off assisted breathing; NO=nitric oxide; and PFT=pulmonary function test.

FIGS. 2a-c are graphs showing various pulmonary parameters measured for 28 days. The data is an aggregate of individual subject data of change from baseline parameters for FiO₂, PEEP, and PaO₂/FiO₂ ratio through day 28. The abbreviations in the graph include: FiO₂=inspired oxygen concentration; INO=inhaled nitric oxide; PaO₂=partial pressure of arterial oxygen; PEEP=positive-end expiratory pressure; and PF=PaO₂/FiO₂ ratio.

FIG. 3 is a graph of the results from pulmonary function tests (mean predicted) at 6 months for various pulmonary parameters. The abbreviations include: FEF=forced expiratory flow; FEF_{25-75%}=FEF from 25% to 75% of FVC; FEV₁=forced expiratory volume in 1 second; FRC=functional residual capacity; FVC=forced vital capacity; TLC=total lung capacity. Statistically significant results are indicated, with a p<0.05, treatment versus placebo.

### DETAILED DESCRIPTION

The present invention is directed to the unexpected finding that short term treatment of ARDS using inhaled nitric oxide gas improves chronic pulmonary function in ARDS survivors.

### DEFINITIONS

As used herein the following terms shall have the definitions set forth below.

The term therapeutic composition may be used interchangeably with the term “device”. The device designation as defined herein is in concurrence with the Food and Drug Administration's (FDA) definition of a device: A device is defined as an instrument, apparatus, implement, machine, contrivance, implant, in vitro reagent, or other similar or related article, including a component part, or accessory which is:
recognized in the official National Formulary, or the United States Pharmacopoeia, or any supplement to them,
intended for use in the diagnosis of disease or other conditions, or in the cure, mitigation, treatment, or prevention of disease, in man or other animals, or
intended to affect the structure or any function of the body of man or other animals, and which does not achieve any of its primary intended purposes through chemical action within or on the body of man or other animals and which is not dependent upon being metabolized for the achievement of any of its primary intended purposes.

The term device is also meant to include the presently claimed composition.

As used herein, the term “treating” refers to the treatment of a disease or condition of interest in a patient (e.g., a mammal) having the disease or condition of interest, and includes, for example one or more of the following:
(i) preventing the disease or condition from occurring in a mammal, in particular, when such mammal is predisposed to the condition but has not yet been diagnosed as having it;
(ii) inhibiting the disease or condition (i.e., arresting its development);
(iii) reducing the extent of disease or condition (i.e., causing regression of the disease or condition); or
(iv) ameliorating the symptoms resulting from the disease or condition (i.e., relieving pain without addressing the underlying disease or condition).

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, “short term treatment” refers to treatment periods up to one month, two months or three months.

As used herein, “chronic treatment” refers to treatment periods of greater than three months.

As used herein, the term “patient” refers to an animal, human or non-human, to whom treatment according to the methods of the present invention is provided. Veterinary applications are anticipated by the present invention. The term “patient” includes but is not limited to birds, reptiles, amphibians, and mammals, e.g., humans, other primates, pigs, rodents such as mice and rats, rabbits, guinea pigs, hamsters, cows, horses, cats, dogs, sheep and goats.

As used herein, the term “subject” is used interchangeably with “patient”.

As used herein, the term “administering” refers to any mode of transferring, delivering, introducing or transporting the therapeutic composition, device or other agent to a subject. Administration of the therapeutic composition, device or other agent may be conducted concurrently or sequentially in time. Additionally, administration of the therapeutic composition, device and other agent(s) may be via the same or different route(s).

As used herein, the term “effective amount” refers to that amount of which, when administered to a patient (e.g., a mammal) for a period of time is sufficient to cause an intended effect or physiological outcome. The amount of therapeutic composition which constitutes an “effective amount” will vary depending on the condition and its severity, the manner of administration, and the patient (e.g., the age of the mammal to be treated), but can be determined routinely by one of ordinary skill in the art having regard to his own knowledge and to this disclosure.

For example, in one embodiment, the term “effective amount” refers to the amount that can achieve a measurable result. In one embodiment, an “effective amount” is, for example, an amount that when administered to a human subject in need of medical treatment in a controlled Phase 2 or Phase 3 clinical trial produces a statistically significant benefit on a predefined clinical endpoint.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutical composition” refers to a formulation of a compound and a medium generally accepted in the art for the delivery of the biologically active compound to mammals, e.g., humans. Such a medium includes all pharmaceutically acceptable carriers, diluents or excipients therefore.

As used herein, the term “indications” includes, ischemia-reperfusion injury including, but not limited to, pulmonary disease, acute lung injury and acute respiratory distress syndrome (ARDS).

The methods and compositions of the present invention may be used to treat or prevent a variety of diseases and disorders, including any disease or disorder that has been treated using any of a gaseous form of nitric oxide, a liquid nitric oxide composition or any medically applicable useful form of nitric oxide, including any described in U.S. Pat. No. 6,103,275.

Diseases, disorders, and conditions that may benefit from treatment with, or are associated with, nitric oxide, nitric oxide precursors, analogs, or derivatives thereof, include elevated pulmonary pressures and pulmonary disorders associated with hypoxemia (e.g., low blood oxygen content compared to normal, i.e., a hemoglobin saturation less than 95% and a Paₒ₂ less than 90 in arterial blood in someone breathing room air) and/or smooth muscle constriction, including pulmonary hypertension, acute respiratory distress syndrome (ARDS), diseases of the bronchial passages such as asthma and cystic fibrosis, other pulmonary conditions including chronic obstructive pulmonary disease, adult respiratory distress syndrome, high-altitude pulmonary edema, chronic bronchitis, sarcoidosis, cor pulmonale, pulmonary embolism, bronchiectasis, emphysema, Pickwickian syndrome, and sleep apnea.

Additional examples of conditions associated with nitric oxide or nitric oxide related treatments include cardiovascular and cardio-pulmonary disorders, such as angina, myocardial infarction, heart failure, hypertension, congenital heart disease, congestive heart failure, valvular heart disease, and cardiac disorders characterized by, e.g., ischemia, pump failure and/or afterload increase in a patient having such disorder, and artherosclerosis. Nitric oxide related treatments may also find use in angioplasty.

Additional examples include blood disorders, including those blood disorders ameliorated by treatment with NO or related molecules, i.e., where NO would change the shape of red blood cells to normal or restore their function to normal or would cause dissolution of blood clots. Examples of blood disorders include, e.g., sickle cell disease and clotting disorders including disseminated intravascular coagulation (DIC), heart attack, stroke, and Coumadin-induced clotting caused by Coumadin blocking protein C and protein S, and platelet aggregation. Additional examples include such conditions as hypotension, restenosis, inflammation, endotoxemia, shock, sepsis, stroke, rhinitis, and cerebral vasoconstriction and vasodilation, such as migraine and non-migraine headache, ischemia, thrombosis, and platelet aggregation, including preservation and processing of platelets for transfusions and perfusion technologies, diseases of the optic musculature, diseases of the gastrointestinal system, such as reflux esophagitis (GERD), spasm, diarrhea, irritable bowel syndrome, and other gastrointestinal motile dysfunctions, depression, neurodegeneration, Alzheimer's disease, dementia, Parkinson's disease, stress and anxiety. Nitric oxide and nitric oxide related treatments may also be useful in suppressing, killing, and inhibiting pathogenic cells, such as tumor cells, cancer cells, or microorganisms, including but not limited to pathogenic bacteria, pathogenic mycobacteria, pathogenic parasites, and pathogenic fungi. Examples of microorganisms include those associated with a respiratory infection within the respiratory tract.

As used herein, the term “tissue” refers to any mammalian body tissue, desirably a human body tissue, including damaged tissue. A body tissue, according to the teachings to the present invention, may be, but is not limited to, muscle tissue, particularly cardiac tissue and, more particularly, myocardial tissue, such as left ventricular wall myocardial tissue.

As used herein, the term “damaged tissue” refers to any damaged mammalian body tissue, including, for example, damaged pulmonary tissue, and particularly, damaged lung tissue.

As used herein, the term “liquid mixture” refers to a composition which is freely flowable and which includes a liquid. In some embodiments, the composition of the liquid mixture may include a mixture of two or more liquids or a mixture of a liquid and a solid. In other embodiments, the liquid mixture includes a liquid but not a solid or only a negligible amount of a solid. Desirably, the liquid mixtures are solutions.

### Gases and Detection of Gases

Methods for safe and effective administration of NO by inhalation are well known in the art. See, e.g., Zapol, U.S. Pat. No. 5,570,683; Zapol et al., U.S. Pat. No. 5,904,938; Frostell et al., 1991, Circulation 83:2038-2047. NO for inhalation is available commercially (INOmax®, INO Therapeutics, Inc., Clinton, N.J.). In the present invention, NO inhalation preferably is in accordance with established medical practice.

A suitable starting dosage for NO administered by inhalation is 20 ppm. See, e.g., INOmax®, package insert (www.inotherapeutics.com). However, dosage can vary, e.g., from 0.1 ppm to 100 ppm, depending on the age and condition of the patient, the disease or disorder being treated, and other factors that the treating physician may deem relevant. Preferably, the lowest effective dose is inhaled. To arrive at the lowest effective dosage empirically, administration can be commenced at 20 ppm and then decreased gradually until vasodilator efficacy is lost. Where 20 ppm is deemed an insufficient inhaled dose, NO dosage may be increased gradually until vasodilator effectiveness is observed. Such adjustment of dosage is routine for those of skill in the art.

Nitric oxide may be administered as either a gas or a liquid. In addition, nitric oxide may be directly administered or provided in the form of a prodrug, metabolite or analog, including prodrug forms that release nitric oxide (see U.S. Pat. No. 7,122,529). For instance, a nitric oxide producing compound, composition or substance may undergo a thermal, chemical, ultrasonic, electrochemical, metabolic or other reaction, or a combination of such reactions, to produce or provide nitric oxide, or to produce its chemical or biological effects. Thus, certain embodiments of the present invention include various nitric oxide and nitric oxide prodrugs, including any nitric oxide producing compound, composition or substance. Certain embodiments of the present invention are directed to nitric oxide precursors and catalysts, such as L-arginine, and analogs and derivatives thereof, and nitric oxide synthases (NOS), and mutants/variants thereof.

Various embodiments of the present invention are directed to nitric oxide donors or analogs, which generally donate nitric oxide or a related redox species and more generally provide nitric oxide bioactivity. Examples of nitric oxide donors or analogs include ethyl nitrite, diethylamine NONOate, diethylamine NONOate/AM, spermine NONOate, nitroglycerin, nitroprusside, NOC compounds, NOR compounds, organic nitrates (e.g., glycerin trinitrate), nitrites, furoxan derivatives, N-hydroxy (N-nitrosamine) and perfluorocarbons that have been saturated with NO or a hydrophobic NO donor.

Additional examples of nitric oxide donors or analogs include S-nitroso, O-nitroso, C-nitroso and N-nitroso compounds and nitro derivatives thereof, such as S-nitrosoglutathione, S-nitrosothiols, nitroso-N-acetylpenicillamine, S-nitroso-cysteine and ethyl ester thereof, S-nitroso cysteinyl glycine, S-nitroso-gamma-methyl-L-homocysteine, S-nitroso-L-homocysteine, S-nitroso-gamma-thio-L-leucine, S-nitroso-delta-thio-L-leucine, S-nitrosoalbumin, S-Nitroso-N-penicillamine (SNAP), glyco-SNAPs, fructose-SNAP-1. Further examples of nitric oxide donors or analogs include metal NO complexes, isosorbide mononitrate, isosorbide dinitrate, molsodomines such as Sin-1, streptozotocin, dephostatin, 1,3-(nitrooxymethyl)phenyl 2-hydroxybenzoate and related compounds (see U.S. Pat. No. 6,538,033); NO complexes with cardiovascular amines, such as angiopeptin, heparin, and hirudin, arginine, and peptides with an RGD sequence (See U.S. Pat. No. 5,482,925); diazeniumdiolates such as ionic diazeniumdiolates, O-derivatised diazeniumdiolates, C-based diazeniumdiolates, and polymer based diazeniumdiolates.

NO is soluble in water up to a concentration of about 2 millimolar (2 mM) at STP. However, the liquid can be any liquid known to those of skill in the art to be suitable for administration to patients (see, for example, Oxford Textbook of Surgery, Morris and Malt, Eds., Oxford University Press, 1994). In certain embodiments, formulations of nitric oxide suitable for administration according to embodiments of the present invention are liquid solutions. Such solutions may comprise water, dextrose, or saline, polymer-bound compositions dissolved in diluents; other aqueous or nonaqueous solvents, such as vegetable oil, synthetic aliphatic acid glycerides, esters of higher aliphatic acids or propylene glycol, including the addition of conventional additives such as solubilizers, isotonic agents, suspending agents, emulsifying agents, stabilizers and preservatives; capsules, sachets or tablets, each containing a predetermined amount of the nitric oxide; solids or granules; suspensions in an appropriate liquid; suitable emulsions; and gases and/or aerosols, for example, as used in inhalation and nebulizer therapy (see, e.g, U.S. Pat. Nos. 5,823,180 and 6,314,956).

In particular embodiments, the present invention includes aerosol formulations, which may include aqueous solutions, lipid soluble aqueous solution, and micronized powders. In certain embodiments the aerosol particle size is between about 0.5 micrometers and about 10 micrometers. Aerosols may be generated by a nebulizer or any other appropriate means.

With respect to gas formulations, those compounds/compositions that are either normally gases or have been otherwise converted to gases may be formulated for use by dilution in nitrogen and/or other inert gases and may be administered in admixture with oxygen, air, and/or any other appropriate gas or combination of multiple gases at a desired ratio. Dilution, for example, to a concentration of 1 to 100 ppm is typically appropriate. In particular embodiments, nitric oxide is used in the range of 10-80 ppm mixed into air.

In one embodiment, nitric oxide and oxygen are generally administered to a patient by diluting a nitrogen-nitric oxide concentrate gas containing about 1000 ppm nitric oxide with oxygen or oxygen-enriched air carrier gas to produce an inhalation gas containing nitric oxide in the desired concentration range (usually about 0.5 to 200 ppm, based on the total volume of the inhalation gas) (see: U.S. Pat. No. 5,692,495).

Polymer-bound compounds/compositions of the present invention may also be used; such compositions are capable of releasing nitric oxide, donors, analogs, precursors, etc., in an aqueous solution and preferably release nitric oxide, etc., under physiological conditions. Any of a wide variety of polymers can be used in the context of the present invention. It is only necessary that the polymer selected is biologically acceptable. Illustrative of polymer suitable for use in the present invention include polyolefins, such as polystyrene, polypropylene, polyethylene, polytetrafluorethylene, polyvinylidene difluoride, and polyvinylchloride, polyethylenimine or derivatives thereof, polyethers such as polyethyleneglycol, polyesters such as poly(lactide/glycolide), polyamides such as nylon, polyurethanes, biopolymers such as peptides, proteins, oligonucleotides, antibodies and nucleic acids, starburst dendrimers, and the like.

The amount of the compounds/compositions of the present invention to be used as a therapeutic agent, of course, varies according to the compounds/compositions administered, the type of disorder or condition encountered and the route of administration chosen. The compositions of the present invention may be prepared for pharmaceutical administration by methods and with excipients generally known in the art. (Remington's Pharmaceutical Sciences (2005); 21^{st} Edition, Troy, David B. Ed. Lippincott, Williams and Wilkins).

A suitable dosage is about 0.01 to 10.0 mg/kg of body weight/day. The preferred dosage is, of course, that amount just sufficient to treat a particular disorder or condition and would preferably be an amount from about 0.05 to 5.0 mg/kg of body weight/day.

When nitric oxide is administered as a gas, a suitable dosage is thought to be between 1 ppm (parts per million) and 1000 ppm, preferentially between 5 ppm and 200 ppm.

In certain embodiments, the suitable dosage is in the range of 5 ppm-10 ppm, 10 ppm-20 ppm, 20 ppm-50 ppm, 50 ppm-100 ppm or more.

In certain embodiments, the amount of or effective compounds/compositions that is provided to a subject can be about, at least, at least about, or at most about any value in the range of 1 to 100 mg, mg/kg, or mg/m² in increments of one, for example, 1, 2, 3, 4 . . . 97, 98, 99, 100 mg, mg/kg, or mg/m², and any value in the range of 100 to 1000 mg, mg/kg, or mg/m² in increments of 10, for example 110, 120, 130 . . . 980, 990 and 1000 mg, mg/kg, or mg/m², or any range derivable therein. Alternatively, the amount may be expressed as any value in the range of 1 to 100 mM or M in increments of one, for example, 1, 2, 3, 4 . . . 97, 98, 99, 100 mM or M, and any value in the range of 100 to 1000 mM or M in increments of 10, for example 110, 120, 130 . . . 980, 990 and 1000 mM or M, or any range derivable therein. In various embodiments of the present invention, a subject is exposed to the compositions of the current invention for about, at least, at least about, 1-24 hours, 1-30 days or more, and any range or combination therein.

All of the U.S. patents, U.S. patent application publications, U.S. patent applications, foreign patents, foreign patent applications and non-patent publications referred to in this specification, are incorporated herein by reference, in their entirety.

### EXAMPLES

### Example 1

### Introduction

Inhaled nitric oxide (iNO) is a vasodilator indicated for treatment of term and near-term neonates with hypoxic respiratory failure associated with clinical or echocardiographic evidence of pulmonary hypertension. In these patients, iNO has been shown to improve oxygenation and reduce the need for extracorporeal membrane oxygenation therapy. NO binds to and activates cytosolic guanylate cyclase, thereby increasing intracellular levels of cyclic guanosine 3′,5′-monophosphate (cGMP). This, in turn, relaxes vascular smooth muscle, leading to vasodilatation. Inhaled NO selectively dilates the pulmonary vasculature, with minimal systemic vasculature effect as a result of efficient hemoglobin scavenging. In acute lung injury (ALI) and acute respiratory distress syndrome (ARDS), increases in partial pressure of arterial oxygen (PaO₂) are believed to occur secondary to pulmonary vessel dilation in better-ventilated lung regions. As a result, pulmonary blood flow is redistributed away from lung regions with low ventilation/perfusion ratios toward regions with normal ratios.

The incidence of ARDS has been estimated to be approximately 75 cases per 100,000 population, although this figure is impacted by ambiguity in the causes and manifestations of ARDS. Mortality rates in ARDS are substantial, with estimates ranging from 34% to 68%, highlighting the need for effective treatment.

Many pharmacologic treatments have been investigated in ARDS patients, including alprostadil, acetylcysteine, corticosteroids, surfactant, dazoxiben, and acyclovir. All studies to date have focused on mortality as the primary endpoint. A meta-analysis of trials completed through 2004 indicated limited mortality benefit with any of the above-mentioned treatments.

Patients surviving an episode of ARDS may have long-term obstructive and restrictive pulmonary abnormalities as well as pulmonary gas exchange impairment. These long-term effects may contribute to decreased quality of life (QoL), repeatedly demonstrated by ARDS survivors. The importance of long-term effects that remain following an ARDS episode has recently emerged, with clinicians noting that assessing short-term survival of ARDS is only part of its clinical impact. Therefore, treatments provided in the intensive care unit (ICU) that improve long-term ARDS outcomes (without improving immediate survival) and clinical studies examining treatment effects on later outcomes may be relevant.

### Study

A large-scale, randomized, blinded, placebo-controlled study was carried out in the Intensive Care Units (ICUs) of 46 US hospitals to evaluate the efficacy of low-dose (5 ppm) iNO in 385 patients with moderately severe Acute Lung Injury (ALI). The primary endpoint of this study was number of days alive and off assisted breathing. Results of an intent-to-treat analysis revealed that inhaled NO (iNO) had no significant benefit versus control (nitrogen gas) as it related to mortality, days alive and off assisted breathing, or days alive and meeting oxygenation criteria for extubation. However, iNO treatment did result in a significant increase (p<0.05) in partial pressure of arterial oxygen (PaO₂) during the initial 24 hours of treatment that resolved by 48 hours.

### Safety Results

Safety results for the initial 28-day study period have been reported and are summarized briefly here. A total of 630 adverse events (AEs) were reported for patients treated with iNO versus 666 events for those who received placebo. Respiratory system AEs occurred in 51% versus 61% of patients receiving iNO and placebo, respectively, primarily due to higher frequencies of pneumonia, pneumothorax, and apnea in the placebo group. Frequency of other AEs was similar in both groups.

The present analysis was developed a priori as part of the original study protocol and carried out to assess long-term pulmonary function differences between iNO and placebo at 6 months post-treatment. This study was the first prospective long-term analysis of pulmonary function in ARDS survivors participating in a randomized interventional clinical trial comparing iNO and placebo.

### Methods

The protocol, amendments to the protocol, and local Informed Consent Forms were reviewed and approved by each of the participating hospitals' Institutional Review Board prior to the initiation of patient accrual. Inclusion and exclusion criteria and treatment details for this analysis are described elsewhere; they are summarized briefly here.

### Patients

Patients had moderately severe acute lung injury (ALI), defined by a modification of American-European Consensus Conference criteria (PaO₂/inspired oxygen concentration [FiO₂] ratio of ≦250 mm Hg), due to causes other than severe sepsis. Patients with evidence of non-pulmonary system failure at the time of randomization and sepsis-induced ARDS were excluded. Patients were also excluded if they had sustained hypotension requiring vasopressor support, hemodynamic profiles supporting severe sepsis, severe head injury, severe burns, or evidence of other significant organ system dysfunction at baseline.

### Treatment

Patients were randomly assigned to receive either inhaled placebo gas (nitrogen) or 5 ppm of iNO (INO Therapeutics Inc., Port Allen, La.). All patients, healthcare professionals, and investigators were blinded to the assigned treatment. Inhaled NO was administered via INOvent® delivery system (Datex-Ohmeda, Madison, Wis.) that blended treatment gas (nitrogen or NO at 100-ppm balance nitrogen) 1:20 with ventilator gases to achieve a target ppm value in the inspiratory limb of the ventilator.

All patients using the iNO delivery system received mechanical ventilatory support. Treatment continued with active or placebo gas until one of the following criteria were met: [1] end of trial (28 days); [2] death; or [3] adequate oxygenation (arterial oxygen saturation by pulse oximetry [SpO₂]≧92% or PaO₂ of ≧63 mm Hg) without treatment gas at ventilator settings of FiO₂≦0.4 and positive end-expiratory pressure (PEEP) of ≦5 cm H₂O. Decreases in treatment gas continued in 20% decrements (titrated down by 1 ppm for inhaled NO) every 30 minutes until either the treatment gas concentration reached 0% or oxygenation criteria were not satisfied. If oxygenation criteria were not met, treatment gas concentration was titrated up until they were again achieved. Increments of upward titration were determined by the clinician, based on degree of arterial desaturation.

### Respiratory Parameters Measured During Hospitalization

Baseline oxygenation measures included PaO₂, arterial partial pressure of CO₂ (PaCO₂), SpO₂, FiO₂, PEEP, PaO₂/FiO₂ ratio, ventricular rate, tidal volume, and mean airway pressure. Respiratory parameters (FiO₂, PEEP, and PaO₂/FiO₂ ratio) were recorded on case report forms every 12 hours during mechanical ventilation.

### Long-Term Pulmonary Function Measures

Pulmonary function testing (PFT) at 6 months post-treatment was required in both iNO- and placebo-treated patients as part of the original study design. PFTs included forced expiratory volume in 1 second (FEV₁), FEV₁% predicted, forced vital capacity (FVC), FVC % predicted, the FEV₁/FVC ratio, FEV₁/FVC ratio % predicted, forced expiratory flow (FEF) from 25% to 75% of FVC (FEF_{25-75%}), FEF_{25-75%}% predicted, functional residual capacity (FRC), FRC % predicted, total lung capacity (TLC), TLC % predicted, CO diffusion, and CO diffusion % predicted.

### Statistical Methods

All between-group differences in PFT results were evaluated using the Wilcoxon rank sum test. Between-group differences in baseline clinical and demographic characteristics were assessed with the Fisher's exact test and the chi-square test for categorical variables and with the Wilcoxon rank sum test for continuous variables. Baseline oxygenation and respiratory/oxygenation parameters in the two groups were compared using Wilcoxon rank sum tests. The areas under the curve (AUCs) of FiO₂, PEEP, and PaO₂/FiO₂ ratio were calculated using the trapezoidal rule. The null hypothesis that the respective AUCs were normally distributed was rejected employing the Shapiro-Wilk test. A Wilcoxon rank sum test was utilized to assess the differences in each median AUC between treatment groups. A p value <0.05 was considered significant.

### Results

### Demographics and Baseline Characteristics

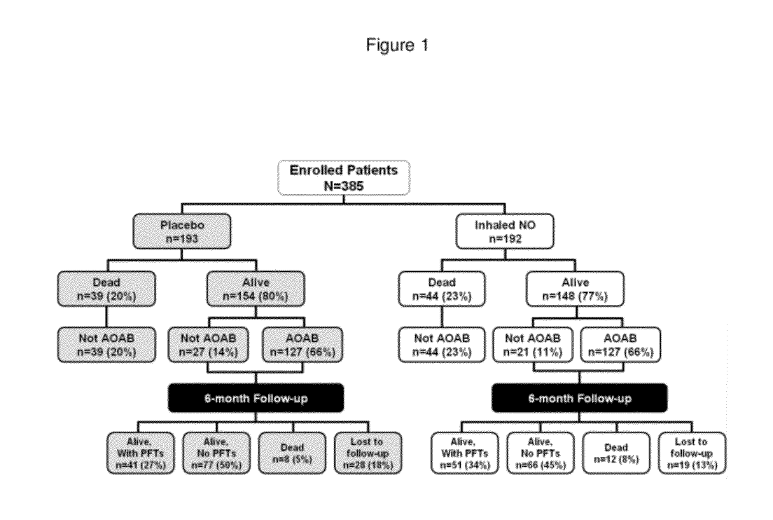

A total of 92 of the 302 survivors (30%) were capable of and participated in the 6-month follow-up pulmonary function analysis (iNO, n=51; placebo, n=41). The balance of surviving subjects was lost to follow-up for reasons unknown, and PFT data are not available. Final disposition of all subjects in the original study and 6-month follow-up is shown in FIG. 1. Baseline patient characteristics are summarized in Table 1. Patients in the two treatment groups were well matched for all demographic variables. The only significant between-group difference was for weight (76.35±19.16 kg [mean±SD] versus 85.67±24.10 kg for iNO and placebo, respectively; p=0.0489). There were no significant differences between groups with respect to ARDS etiology. There were no differences between groups with respect to severity of illness, frequency of co-morbid chronic respiratory conditions (i.e., asthma, chronic obstructive pulmonary disease, or other obstructive or restrictive lung disease), or use of inhaled corticosteroids. More subjects had a history of tobacco use in the iNO group (26 versus 17, p=0.41).

### Baseline Oxygenation Parameters

Baseline oxygenation parameters, including PaO₂, PaCO₂, SpO₂, FiO₂, PEEP, and PaO₂/FiO₂ ratio are summarized in Table 2. The patients included in this analysis were severely ill with mean baseline PaO₂/FiO₂ ratios of 140.5±43.4 (iNO) and 136.1±40.4 (placebo). Except for a clinically insignificant difference in SpO₂, there were no significant between-group differences with respect to baseline oxygenation parameters.

### Baseline Respiratory Parameters

Baseline respiratory parameters, including ventilator rate, tidal volume, and mean airway pressure are summarized in Table 3. There were no significant differences between groups for any of these measures.

### Respiratory Parameters During Mechanical Ventilation

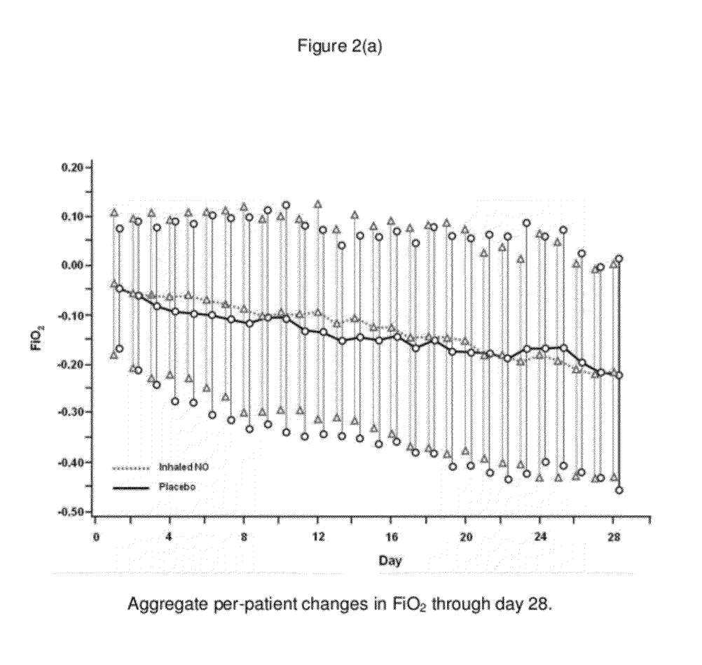

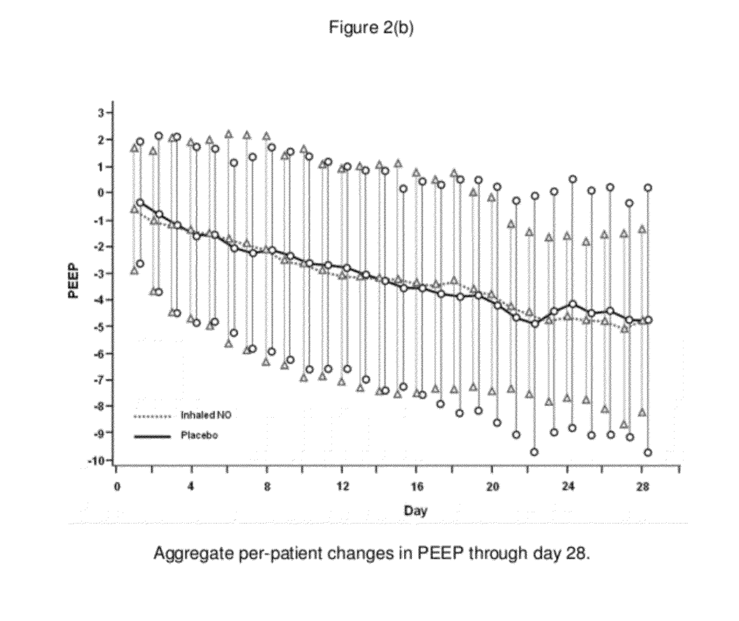

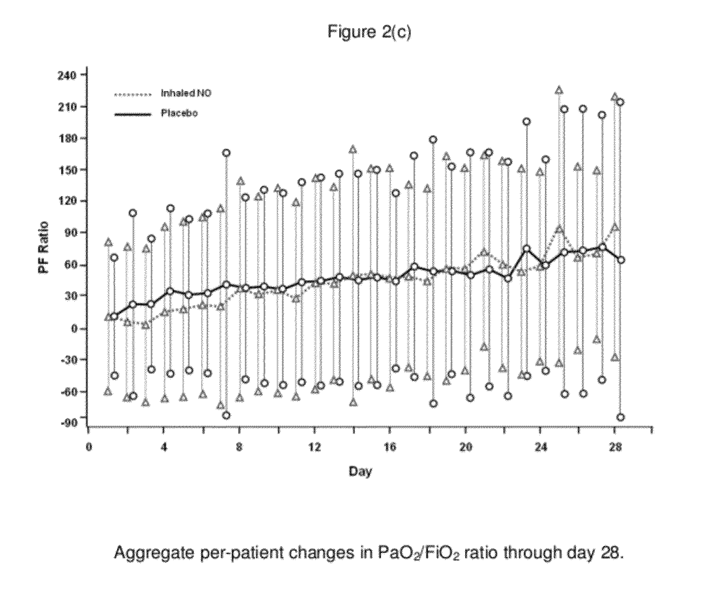

There were no significant differences between groups for aggregate per-patient changes from baseline parameters in supplemental oxygen, PEEP, or PaO₂/FiO₂ ratio (FIG. 2a-c). However, when calculating the duration of exposure over the length of mechanical ventilation for total FiO₂ (6.3±4.5 days versus 7.6±4.7 days for iNO and placebo groups, respectively; p=0.151), total PEEP (96.3±75.9 versus 113.4±81.1 mm Hg, p=0.261) and total PaO₂/FiO₂ ratio (2637±1729 versus 2950±1774, p=0.358), the iNO group had less cumulative exposure to all three variables (Table 4).

### Pulmonary Function Tests at 6 Months

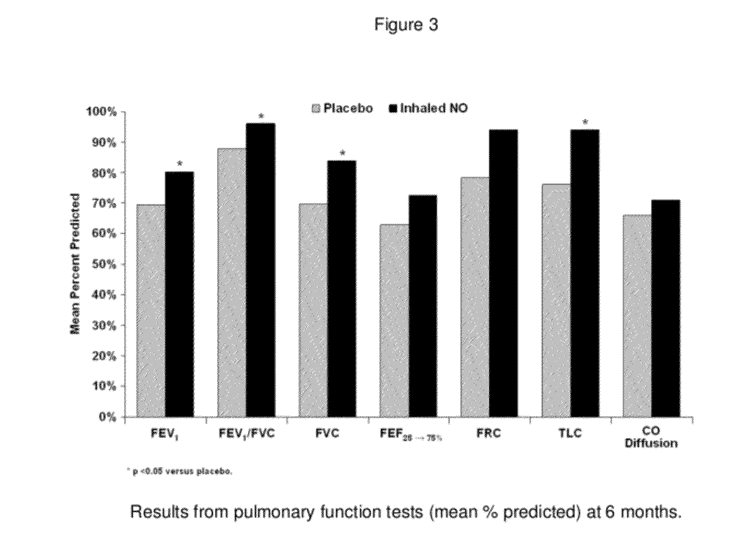

Results for PFTs at 6 months post-treatment with placebo or iNO are summarized in Table 5 and FIG. 3. Study results indicated significantly greater values for patients treated with iNO versus placebo for FEV₁% predicted (p=0.042), FVC % predicted (p=0.019), FEV₁/FVC % predicted (p=0.033), TLC (p=0.026), and TLC % predicted (p<0.001).

### SUMMARY

Clinical trials evaluating numerous interventions have repeatedly failed to demonstrate significant benefit in decreasing mortality in ARDS patients. Other endpoints, such as long-term morbidity or a shift of focus to short- and long-term respiratory changes in survivors of ARDS, may be important when evaluating established and emerging ARDS treatments. In a study evaluating 50 long-term ARDS survivors, assessed a median of 5.5 years after ICU discharge, 54% had impairment (defined as <80% predicted value) in at least one pulmonary function measure including decreases in FEV1/FVC ratio consistent with airflow obstruction (n=16), residual volume (n=14), TLC (n=10), and diffusing capacity (n=8). Seven patients had multiple pulmonary function abnormalities; health-related QoL was also significantly decreased.

This is the first prospective study to evaluate iNO effects on long-term pulmonary function in ARDS patients. Previous trials of iNO in ARDS patients focused on shorter-term outcomes, primarily mortality, and failed to demonstrate significant benefit. The original clinical trial, as well as a meta-analysis of 12 randomized controlled trials in ALI or ARDS patients indicated no significant benefit of iNO in decreasing mortality and only transient effects on physiological endpoints, such as PaO2/FiO2 ratio.

Our results demonstrate an association between iNO and better PFT results at 6 months post-treatment. The clinical significance of longer-term lung function and QoL in ARDS survivors has been examined in other studies. Results from one long-term follow-up of ARDS survivors indicated that both FEV1 and FVC at 12 months post-episode were correlated with the physical function domain of two validated QoL questionnaires.

Additionally, the cumulated aggregate per-subject values for FiO2, PEEP, and PaO2/FiO2 exposure days, while not reaching statistical significance, were less in the iNO-treated patients compared with placebo. Taken together, the differences in 6-month PFTs, as well as the aggregate oxygenation exposure data, suggest a potential positive long-term physiologic effect of iNO on lung function in ARDS survivors.

In addition to the decreased duration of FiO2 and PEEP exposure during the 28-day trial, several other potential mechanisms may underlie possible longer-term benefit of iNO on pulmonary function. ARDS is associated with pronounced elevations in multiple inflammatory markers, and several studies have suggested that this may be attenuated by iNO. Studies with experimental animals and ARDS patients have shown that iNO significantly decreases pulmonary concentrations of interleukin (IL)-8 and neutrophils. as well as significantly inhibiting the formation of platelet-leukocyte aggregates (an effect correlated with an NO-dependent inhibition of platelet P-selectin expression). This may potentially lead to improvement of microcirculation in vascular beds, including muscle. In another study conducted in ARDS patients, iNO was found to significantly decrease H2O2 production and β2-integrin CD11b/CD18 expression by polymorphonuclear leukocytes. In addition, iNO decreased IL-6 and IL-8 concentrations in BAL fluid. There is also evidence that NO inhibits activation of protease-activated receptor-1, which was shown to increase vascular permeability and edema when stimulated in experimental animals.

Inhaled NO did not improve short-term mortality in patients with ARDS, despite transient physiologic benefit. Perhaps the simplest explanation for this is that these patients primarily die as a result of multiple organ failure or sepsis, rather than refractory hypoxemia. Thus, changes in oxygenation sustained for only 24 hours with iNO are not sufficient to alter mortality.

### Limitations

This analysis had limitations, primarily: 1) the large percentage of subjects lost to follow-up who did not have PFTs performed at 6 months; and 2) the inability to obtain premorbid PFTs. Even though the former constituted a protocol violation, the reasons for this occurring are not available, potentially influencing the results via significant bias or confounding. Additionally, while the lack of premorbid PFTs would have provided valuable insight, from a practical study standpoint, obtaining these values was not possible. The fact that the baseline characteristics between groups were very similar, especially with respect to severity of illness, co-morbid chronic respiratory conditions, and use of inhaled corticosteroids, suggests that these potential influences may have been minimized.

There was a small but statistically significant difference in SpO2 that favored iNO group at baseline. Tidal volume was higher, and ventilator rate and mean airway pressure were lower in the patients receiving iNO; however, there was no consistent pattern of these small, non-significant differences that would support an influence on pulmonary function at the 6-month follow-up. Finally, it is important to note that the original study's inclusion criteria did not exclude preexisting lung disease, and treatment assignment was not stratified on that basis.

In determining the optimal approach to future trials, a number of study design parameters should be considered. Additional baseline patient characteristics (eg, type and burden of tobacco exposure between groups, bronchodilators/inhaled corticosteroids) and analysis for premorbid PFTs, if possible, are important in minimizing potential survivor selection and better functioning patient biases. From a results standpoint, overall duration on mechanical ventilation, incidence of lower respiratory tract infections, and measurements of change in mean airway pressure, plateau pressure, and tidal volume will provide enhanced perspectives on clinical benefit in this area.

While current clinical research regarding ARDS treatment has focused on mortality and short-term effects of treatment, it is important to consider chronic lung effects in ARDS survivors, which are a major cause of long-term morbidity and reduction of QoL in this population. Results from this 6-month analysis show that ARDS survivors who received iNO had significantly better PFT parameters versus placebo.

These results support consideration of further clinical trials to determine the longer-term effects of iNO on the incidence and severity of chronic lung disease in ARDS patients. Additional outcomes that should be explored include measures of health-related QoL, healthcare utilization, and overall patient management cost.

### TABLES

Table 1 is a summary of baseline demographic and clinical characteristics of the study group.

Table 2 is a summary of baseline oxygenation parameters of the study group (placebo versus treated).

Table 3 is a summary of baseline respiratory parameters of the study group (placebo versus treated).

Table 4 is a summary of the duration of exposure parameters during gas administration.

Table 5 is a summary of pulmonary function test results of the study subjects at 6 months.

TABLE 1
Baseline demographic and clinical characteristics.

| Parameter | | Placebo | | Inhaled NO | | P Value |
|---|---|---|---|---|---|---|
| Age, y | N | 41 | | 51 | | |
| | Mean ± SD | 47.8 ± 16.7 | | 45.3 ± 15.3 | | 0.494 |
| | Range | 18.4-84.0 | | 16.8-77.9 | | |
| Sex, n (%) | Male | 19 | (46%) | 25 | (49%) | 0.836 |
| | Female | 22 | (54%) | 26 | (51%) | |
| Race, n (%) | Caucasian | 35 | (85%) | 42 | (82%) | 0.847 |
| | Black | 4 | (10%) | 5 | (10%) | |
| | Other | 2 | (5%) | 4 | (8%) | |
| Height, cm | N | 39 | | 51 | | |
| | Mean ± SD | 168.7 ± 11.4 | | 169.4 ± 9.2 | | 0.912 |
| Weight, kg | N | 41 | | 51 | | |
| | Mean ± SD | 85.7 ± 24.1 | | 76.4 ± 19.2 | | 0.049 |
| Causes of ARDS,* | | | | | | |
| n (%) | | | | | | |
| Pneumonia | | 20 | (49%) | 15 | (29%) | 0.084 |
| Toxic gas inhalation | | 0 | (0%) | 0 | (0%) | 1.000 |
| Acute pancreatitis | | 1 | (2%) | 3 | (6%) | 0.626 |
| Massive blood | | 5 | (12%) | 10 | (20%) | 0.404 |
| transfusion | | | | | | |
| Fat emboli | | 1 | (2%) | 2 | (4%) | 1.000 |
| Aspiration | | 9 | (22%) | 9 | (18%) | 0.610 |
| pneumonitis | | | | | | |
| Pulmonary contusion | | 6 | (15%) | 12 | (24%) | 0.307 |
| Postpartum ARDS | | 2 | (5%) | 0 | (0%) | 0.196 |
| Multiple trauma | | 14 | (34%) | 15 | (29%) | 0.657 |
| Elective or emergency | | 9 | (22%) | 20 | (39%) | 0.114 |
| surgical procedures | | | | | | |
| Preexisting lung | | 41 | (100%) | 49 | (96%) | 0.501 |
| disease | | | | | | |
| Preexisting steroid use | | 3 | (7%) | 6 | (11.8%) | 0.334 |
| Asthma | | 4 | (10%) | 5 | (10%) | 1.000 |
| COPD | | 6 | (15%) | 6 | (12%) | 0.761 |
| Tobacco use | | 17 | (41%) | 26 | (51%) | 0.405 |
| Other lung disease^{†} | | 10 | (5%) | 8 | (4%) | 0.810 |

ARDS = acute respiratory distress syndrome; COPD = chronic obstructive pulmonary disorder; NO = nitric oxide.
*Patients may have more than one cause of ARDS.
^{†}Patients may have more than one preexisting disease including: cancer, bronchitis, amiodarone toxicity, and status/post lung resection.

TABLE 2
Baseline oxygenation parameters.

| Parameter | Statistics | Placebo | Inhaled NO | P Value |
|---|---|---|---|---|
| PaO₂, mm Hg | N | 41 | 50 | |
| | Mean ± SD | 84.8 ± 21.4 | 90.6 ± 19.1 | |
| | Median | 81 | 86 | 0.068 |
| PaCO₂, mm Hg | N | 41 | 50 | |
| | Mean ± SD | 39.9 ± 7.7 | 40.8 ± 8.4 | |
| | Median | 41 | 39 | 0.728 |
| SpO₂, % | N | 41 | 50 | |
| | Mean ± SD | 95.1 ± 2.6 | 96.5 ± 2.6 | |
| | Median | 96 | 97 | 0.012 |
| FiO₂ | N | 41 | 50 | |
| | Mean ± SD | 0.65 ± 0.13 | 0.68 ± 0.16 | |
| | Median | 1 | 1 | 0.517 |
| PEEP, cm H₂O | N | 41 | 51 | |
| | Mean ± SD | 9.5 ± 1.7 | 9.8 ± 2.5 | |
| | Median | 10 | 10 | 0.748 |
| PaO₂/FiO₂ | N | 41 | 50 | |
| ratio | Mean ± SD | 136.1 ± 40.4 | 140.5 ± 43.4 | |
| | Median | 132 | 130 | 0.774 |

FiO₂ = inspired oxygen concentration; PaCO₂ = arterial pressure of CO₂; PaO₂ = partial pressure of arterial oxygen; PEEP = positive-end expiratory pressure; SpO₂ = pulse oximetric oxygen saturation.

TABLE 3
Baseline respiratory parameters.*

| Parameter | Statistics | Placebo | Inhaled NO | P Value |
|---|---|---|---|---|
| Ventilator rate, | N | 41 | 50 | 0.069 |
| breaths/min | | | | |
| | | 14.6 ± 4.4 | 13.1 ± 4.2 | |
| Tidal volume, mL/kg | N | 39 | 49 | 0.548 |
| | | 9.1 ± 1.7 | 10.3 ± 2.5 | |
| Mean airway pressure, | N | 37 | 46 | 0.488 |
| cm H₂O | | | | |
| | | 18.3 ± 7.1 | 16.9 ± 5.2 | |

*Values are mean ± SD unless otherwise indicated.
NO = nitric oxide.

TABLE 4
Duration of exposure parameters during study gas administration.*

| | Placebo | Inhaled NO | |
|---|---|---|---|
| Parameter | (N = 41) | (N = 51) | P Value |
| Inhaled NO, ppm/d | 0 | 114 ± 102 | NA |
| FiO₂ | 7.6 ± 4.7 | 6.34 ± 4.5 | 0.151 |
| PEEP, mm Hg | 113 ± 81 | 96.33 ± 75.9 | 0.261 |
| PaO₂/FiO₂ ratio | 195 ± 46 | 262 ± 407 | 0.358 |

*Values are mean ± SD unless otherwise indicated.
FiO₂ = inspired oxygen concentration; NO = nitric oxide; PaO₂ = partial pressure of arterial oxygen; PEEP = positive-end expiratory pressure.

TABLE 5
Pulmonary function test results at 6 months.

| Parameter | Statistics | Placebo | Inhaled NO | P Value |
|---|---|---|---|---|
| FEV₁, L | N | 41 | 51 | |
| | | 2.29 (0.71) | 2.64 (0.91) | 0.1161 |
| FEV₁, % predicted | N | 41 | 50 | |
| | | 69.51 (28.97) | 80.23 (21.21) | 0.042 |
| FEV₁/FVC, % | N | 40 | 51 | |
| | | 72.89 (20.20) | 77.45 (15.19) | 0.155 |
| FEV₁/FVC, % predicted | N | 37 | 49 | |
| | | 87.92 (19.77) | 96.14 (13.79) | 0.033 |
| FVC, L | N | 41 | 51 | |
| | | 3.01 (0.96) | 3.36 (1.09) | 0.163 |
| FVC, % predicted | N | 41 | 50 | |
| | | 69.84 (27.40) | 83.78 (19.38) | 0.019 |
| FEF_{25-75%}, L/sec | N | 41 | 51 | |
| | | 12.25 (55.86) | 26.34 (84.50) | 0.121 |
| FEF_{25/75%}, % predicted | N | 41 | 50 | |
| | | 62.96 (36.26) | 72.50 (27.71) | 0.154 |
| FRC, L | N | 33 | 44 | |
| | | 2.64 (0.71) | 3.00 (0.94) | 0.113 |
| FRC, % predicted | N | 32 | 43 | |
| | | 78.19 | 93.98 | 0.109 |
| TLC, L | N | 32 | 44 | |
| | | 4.81 | 5.54 | 0.026 |
| TLC, % predicted | N | 31 | 43 | |
| | | 76.10 | 93.33 | <0.001 |
| CO diffusion, mL/min/mm Hg | N | 33 | 42 | |
| | | 17.87 (6.37) | 18.25 (6.77) | 0.709 |
| CO diffusion, % predicted | N | 32 | 42 | |
| | | 65.96 (23.23) | 71.02 (20.79) | 0.492 |

*Values are mean (SD) unless otherwise indicated.
FEF = forced expiratory flow; FEV₁ = forced expiratory volume in 1 second; FRC = functional residual capacity; FVC = forced vital capacity; TLC = total lung capacity.

What is claimed is:

1. A method of treating a subject with impaired pulmonary function as a result of acute respiratory distress syndrome (ARDS) comprising: administration of a low dose of inhaled nitric oxide (NO) wherein the inhaled NO improves pulmonary function after short term treatment.

2. The method of claim 1, wherein the inhaled NO is administered at a dose of at least 0.1 ppm to 100 ppm

3. The method of claim 1, wherein the inhaled NO is administered at a dose of at least 5 ppm.

4. The method of claim 1, wherein the inhaled NO is administered up to 28 days.

5. The method of claim 1, wherein the impaired pulmonary function is improved for at least six months.

6. The method of claim 1, wherein the treatment improves pulmonary function parameters consisting of total lung capacity, forced expiratory volume and forced vital capacity.

7. The method of claim 1, wherein the treatment period is in the range of one month to seven months.

8. The method of claim 1, wherein the treatment period is in the range of 4 months to six months.

9. The method of claim 1, wherein the treatment period is in the range of 5 months to 7 months.

10. The method of claim 1, wherein the inhaled NO is administered at a dose of at least 5 ppm for up to 28 days wherein the treatment improves pulmonary function consisting of: total lung capacity, forced expiratory volume and forced vital capacity.

11. The method of claim 1, wherein the inhaled NO is administered by a InoMax device.
